(19) United States
(12) Patent Application Publication
(10) Pub. No.: US 2026/0358312 A1
(43) Pub. Date: Nov. 12, 2026

(54) DEVICE AND METHOD FOR QUANTIFYING A TOTAL NITROGEN CONTENT PRESENT IN A SAMPLE OF A POROUS MEDIUM AND/OR DERIVED FROM BIOMASS

(71) Applicant: IFP Energies nouvelles, RUEIL-MALMAISON (FR)

(72) Inventors: Isabelle KOWALEWSKI, RUEIL-MALMAISON CEDEX (FR); Herman RAVELOJAONA, RUEIL-MALMAISON CEDEX (FR); Olivier SISSMANN, RUEIL-MALMAISON CEDEX (FR); Philipp SCHIFFMANN, RUEIL-MALMAISON CEDEX (FR); Sonia NOIREZ, RUEIL-MALMAISON CEDEX (FR); Violaine LAMOUREUX-VAR, RUEIL-MALMAISON CEDEX (FR)

(21) Appl. No.: 19/134,832

(22) PCT Filed: Dec. 8, 2023
(86) PCT No.: PCT/EP2023/084842

§ 371 Date: Jun. 2, 2025

(30) Foreign Application Priority Data
Dec. 19, 2022 (FR) FR2213721

(51) Int. Cl. G01N 31/12; G01N 21/33; G01N 33/00

(52) U.S. Cl. CPC G01N 31/12; G01N 21/33; G01N 33/0037; G01N 33/0054

(57) ABSTRACT

The invention concerns a device and a method for quantifying a total nitrogen content in at least one of a porous medium and a biomass sample. The sample is heated in an inert atmosphere between a temperature ranging between 50 °C and 300 °C and a temperature ranging between 650 °C and 1000 °C, and NH₃, N₂O and N₂ contents, as well as first NO and NO₂ contents are determined; effluents from the first heating are subjected to continuous oxidation at 900 °C at least, and second NO and NO₂ contents are determined; a residue of the sample is heated in an oxidizing atmosphere between a temperature ranging between 50 °C and 300 °C and a temperature of at least 850 °C, and third NO and NO₂ contents are determined. The total nitrogen content corresponds to the sum of the NH₃, N₂O and N₂ contents and of the first, second and third NO and NO₂ contents.

### CROSS-REFERENCE TO RELATED APPLICATIONS

Reference is made to PCT/EP2023/084842 filed Dec. 8, 2023, and French Application No. 2213721, filed Dec. 19, 2022, which are incorporated herein by reference in their entirety.

### BACKGROUND OF THE INVENTION

### Field of the Invention

The present invention concerns the field of quantification of the nitrogen present in a porous medium and/or biomass sample.

### Description of the Prior Art

Applied to soil science, environmental geosciences or agriculture, the present invention can notably allow quantification of the nitrogen present in a soil sample, a superficial formation, or a fertilizer for a soil.

Nitrogen is essential for plant growth. Nitrogen fertilization is necessary for crop development. Thus, nitrogen deficiency jeopardizes crop yield and quality. On the other hand, excess nitrogen is harmful to the environment, notably because, under certain circumstances, nitrogen fertilizers turn into nitrous oxide, a powerful greenhouse gas.

Thus, in addition to the carbon content, the nitrogen content is an indicator providing information on the condition of a soil, in particular within the framework of organic agriculture whose purpose is to reduce or even to eliminate the use of synthetic fertilizers in a context of climate change issues. Note that, although the use of a synthetic nitrogen fertilizer by atmospheric nitrogen fixation led to the so-called “green revolution” in the 1930s, it is precisely the excessive use thereof that leads today to a disastrous environmental effect on the aquatic systems due to the eutrophication process.

The term nitrogen designates two groups: organic nitrogen and inorganic nitrogen. Organic nitrogen is the nitrogen present in any organic compound (such as amino acids, proteins, nitrogen related to decomposing vegetable and animal waste, etc.), and inorganic nitrogen is the nitrogen present in inorganic compounds (such as ammonium (NH₄⁺), nitrates (NO₃⁻), gaseous nitrogen (N₂) and nitrites (NO₂)).

In a soil, nitrogen is predominantly present in organic form (about 95% of the total nitrogen in a soil). These nitrogen forms cannot be directly assimilated by plants (except for urea, a small-size molecule). Organic nitrogen consists of various nitrogen-containing compounds whose mineralization under the effect of microbial activity is very variable and extremely difficult to predict.

In mineral form, nitrogen can be present in nitrates: KNO₃ (niter) and NaNO₃ (Chile saltpeter), ammonium nitrates (the most widely used fertilizers in France), and in urea (nitrogen-containing fertilizers). The mineral nitrogen in soils comprises the nitrogen dissolved in the soil solution or fixed on the clay-humus complex, whose main forms are nitrate (NO₃⁻, nitric nitrogen) and ammonium ions (NH₄⁺, ammoniacal nitrogen). It is in mineral form that plants draw nitrogen. Nitric nitrogen (NO₃) is the most plant available form, and ammoniacal nitrogen mainly results from the hydrolysis of amino acids. Due to its positive charge, NH₄⁺ is fixed in the clay-humus complex of the soil, which limits risks of being carried deep down (by leaching), as well as its immediate availability for plants. The microbial activity of soils consumes ammonium and converts it to nitric nitrogen, it is the nitrification process. In case of leaching or denitrification, the nitrogen is lost to the ecosystem.

Nitrogen may be present in several oxidation states, from the most reduced to the most oxidized volatile gaseous form: NH₃ (ammonia), N₂ (dinitrogen), N₂O, NO, NO₂.

“Total nitrogen” is the sum of the nitrogen forms present in a sample in its organic and/or mineral configuration. The nitrogen stock in the soil changes over time according to three phenomena: mineralization, leaching and vegetation cover development. The soil fertility (or quality) is based among others on a carbon and nitrogen elemental analysis. In addition to the soil texture and structure, the pH value and the pedoclimatic conditions, this fertility (decomposition rate of the organic matter of the soil and nitrogen mineralization rate) depends on the organic content of the soil in C and N (quality and quantity), and on the fertilizer supply and organic or mineral amendments. There are many increasingly varied organic fertilizers, and mere knowledge of the elemental composition of soils (corresponding in principle to the organic C/organic N ratio, which is however often equated with total C/total N) makes it more and more difficult to predict their evolutions in the soil. This C/N ratio provides information on the degree of evolution of the organic matter, the biological activity of the soil and the potential for nitrogen supply by the soil (mineralization). However, the total nitrogen provides no information on the plant-available mineral nitrogen.

The following documents are mentioned in the description:
Jaber A. M. Y., Mehanna N. A., Sultan S. M.; 2009. Determination of Ammonium and Organic Bound Nitrogen by Inductively Coupled Plasma Emission Spectroscopy, Talanta 78 (4-5), pp. 1298-1302, https://doi.org/10.1016/j.talanta.2009.01.060.
Hsieh, Y. P. (2007). A Novel Multielemental Scanning Thermal Analysis (MESTA) Method for the Identification and Characterization of Solid Substances. Journal of AOAC International. 90. 54-9.
Hsieh Y. P., Bugna G. C., 2008. Analysis of Black Carbon in Sediments and Soils Using Multi-Element Scanning Thermal Analysis (MESTA), Organic Geochemistry, Vol. 39 (11), pp 1562-1571, ISSN 0146-6380, https://doi.org/10.1016/j.orggeochem.2008.07.015.

Current soil nitrogen analysis methods are most often carried out on fresh and dried fine soil (particles less than 2 mm). Indeed, without any particular precaution, the amount of nitric nitrogen (NO₃⁻) and ammoniacal nitrogen (NH₄⁺) may change very rapidly (less than 24 hours) by organic matter mineralization. These various nitrogen measurement methods are either physico-chemical or thermal methods.

Physico-chemical methods generally involve standards. For example, the soil total nitrogen (in its ammonium, nitrate, nitrite and organic form) can be measured using the so-called Dumas method (elemental analysis—NF ISO 13,878), which allows to determine the total nitrogen content by dry combustion, more precisely by combustion in the presence of oxygen of the crushed soil in a furnace at 950 °C. Also known is the so-called modified Kjeldahl method (NF ISO 11,261) for determining the total nitrogen (in its ammonium, nitrate, nitrite and organic form, i.e. N-tot.=N-org.+N-NH₄+N−NO₃), which comprises three steps: a) mineralization in form of ammonium nitrogen by H₂SO₄ (which converts the organic matter to NH₄⁺), b) distillation of the NH₃ formed with water vapor, and c) distillate titration using boric acid. The quantities needed for measurement range from 0.1 to 10 g samples. The mineral nitrogen (N—NH₄ and N—NO₃) can be measured in two parts according to the NF ISO 14,256 standard that involves colorimetric methods. More precisely, determination of the nitrates, nitrites and ammonium in field-moist soils is performed by extraction with a potassium chloride solution, followed by spectrophotometric analyses of the different nitrogen types (reading at 543 nm for nitrites and nitrates, and at 660 nm for ammonium). The results for the amounts of total nitrogen and in its various forms after the Kjeldahl method are respectively given in mg/kg on moist soil, mg/kg on dry soil and kg/ha. Calculation of the organic nitrogen can also be obtained by the difference between the total nitrogen and the mineral nitrogen. These measurements obtained according to standardized procedures are generally performed by accredited design offices or laboratories.

Among the known thermal methods, the method described in the document (Jaber et al., 2009) is based on inductively coupled plasma emission spectrometry (also known as ICP-AES) integrating an improved plasma flow generation system. This method allows to obtain total nitrogen contents comparable to those obtained with the standard Kjeldahl method, but it is much more sensitive. It is also more sensitive than the conventional ICP-AES elemental analysis, which has the drawback of generating many matrix interferences.

More recent elemental analyzers are also known, for example the 828 and 928 Series elemental analyzers from LECO (USA) for total nitrogen determination, combining dry combustion under O₂ with detection of the N₂ generated by use of a thermal conductivity detector (TCD) in a He or Ar stream, using a heated catalytic oven (Cu) for reduction of the nitrogen oxides species (NOx) to N₂. According to the sample type (soil, fertilizer, plants), the samples are sifted (0.150 to 0.5 mm) and dried (85 °C to 105 °C) beforehand. The amount of samples ranges from 0.05 g to 2 g, and the method requires ceramic boats. This detector, by measuring only N₂, allows determining the amount of total nitrogen present in a sample using a high-temperature catalytic oven.

Like all the other elemental analyzers described above, these new analyzers improve the Dumas combustion method for total nitrogen measurement by providing reliability, higher analytical performance and ease of use. The advantage of such analyses is that they are faster and they use neither solvent nor pretreatment compared to the Kjeldahl method for total nitrogen measurement. On the other hand, these methods measure the total nitrogen only from one nitrogen form (N₂ for example), whereas the Kjeldahl method measures up to three nitrogen forms to describe the total nitrogen: N-Org., N—NH₄ and N—NO₃.

Also known is a multi-element scanning thermal analysis (MESTA) method described in the documents (Hsieh, 2007; Hsieh and Bugna, 2008), which allows simultaneous determination of the decomposition of carbon, nitrogen and sulfur compounds according to temperature. More precisely, this method is implemented using two ovens: an oven for the sample (that may weigh between 0.3 and 10 mg) programmable from ambient temperature up to 800 °C with a 50 °C/min gradient in a stream of a O₂/extra-pure He mixture (40/60%), and a combustion oven maintained at 1100 °C and constantly purged with 100% ultra-pure O₂. After pyrolysis, the effluents are transported by the carrier gas to the combustion oven where they are oxidized to CO₂, NO₂ and SO₂. The oxidized materials then successively pass through C (infrared type CO₂ analyzer), N (nitrogen chemiluminescence detector NCD) and S (sulfur chemiluminescence detector SCD) detectors. This method measures the total nitrogen content, but from a single nitrogen form (NO₂) obtained only after oxidation. On the other hand, this method provides information on the total nitrogen quality according to temperature.

Various methods are thus used to determine the total nitrogen of samples, from the soil for example. For organic nitrogen, it may be obtained only with the Kjeldahl method or by difference between the total and mineral nitrogen. However, most of these methods are difficult to implement, with complicated procedures, both time and solvent consuming, and associated with problems such as limitation to high nitrogen concentrations, matrix interferences (except for ICP-AES), and a nearly non-existent (except for the MESTA thermal method) dynamic range (continuously measured signal).

### SUMMARY OF THE INVENTION

The present invention allows these drawbacks to be overcome. More precisely, the present invention concerns a device and a method for measuring the total nitrogen content in at least one of a porous medium and biomass sample, by use of heating sequences in an inert atmosphere and in an oxidizing atmosphere whose characteristics allow complete thermal cracking of the various nitrogen compounds contained in at least one of a porous medium and biomass sample, and by use of measurements of the various nitrogen compounds contained in the effluents resulting from these heating sequences.

The present invention allows quantification of the total nitrogen in a single step in a sample, without pretreatment, and with small amounts (from a few mg to a maximum of a hundred mg). The dynamic character (continuously measured signal) provides a second piece of information on the quality of the nitrogen constituents present in a sample according to the thermal stability thereof (continuous recording of the signal over time and temperature). These parameters allow better identification of the various mechanisms (N₂ fixation, nitrification, denitrification) involved during the carbon cycle-related respiration and fermentation processes. Besides, the present invention also provides, due to the detection of several nitrogen compounds, information on the type of nitrogen compounds present in the sample (and not only concerning NO₂ or N₂ according to the analysis method used), while associating it with the thermal stability thereof.

The present invention concerns a device for quantifying a total nitrogen content in at least one of a porous medium and biomass sample, the device being characterized in that it comprises:
means for heating the sample in an inert atmosphere,
means for heating in an oxidizing atmosphere a residue from the means for heating the sample in an inert atmosphere,
means for oxidizing effluents from the means for heating the sample in an inert atmosphere,
means suited for continuous measurement of:
   an amount of NH₃, an amount of NO, an amount of NO₂, an amount of N₂O and an amount of N₂ from the means for heating the sample in an inert atmosphere,
   an amount of NO and an amount of NO₂ from the means for oxidizing effluents from the means for heating the sample in an inert atmosphere,
   an amount of NO and an amount of NO₂ from the means for heating in an oxidizing atmosphere a residue from the means for heating the sample in an inert atmosphere.

According to one implementation of the invention, the means for heating the sample in an inert atmosphere can comprise an inert-atmosphere pyrolysis oven, and the means for heating in an oxidizing atmosphere the residue from the means for heating the sample in an inert atmosphere comprise an oxidation oven.

According to an implementation of the invention, the means for heating the sample in an inert atmosphere and the means for heating in an oxidizing atmosphere the residue from the means for heating the sample in an inert atmosphere can comprise a single pyrolysis oven that can operate in an inert atmosphere and in an oxidizing atmosphere.

According to an implementation of the invention, the device further comprises means for determining a total nitrogen content from continuous measurement of an amount of NH₃, an amount of NO, an amount of NO₂, an amount of N₂O and an amount of N₂ from the means for heating the sample in an inert atmosphere, an amount of NO and an amount of NO₂ from the means for oxidizing effluents from the means for heating the sample in an inert atmosphere, and an amount of NO and an amount of NO₂ from the means for heating in an oxidizing atmosphere a residue from the means for heating the sample in an inert atmosphere. These means for determining a total nitrogen content can comprise IT resources, notably a computer or a server, including at least a processor and a computer memory. According to an implementation of the invention, the means for oxidizing the effluents from the means for heating the sample in an inert atmosphere can comprise an oxidation oven.

According to an implementation of the invention, the means suited for continuous measurement of the amount of NH₃ from the means for heating the sample in an inert atmosphere, and the means suited for continuous measurement of the amount of at least one of NO and the amount of NO₂ from the means for heating the sample in at least one of an inert atmosphere and from the means for oxidizing effluents from the means for at least one of heating the sample in an inert atmosphere and from the means for heating in an oxidizing atmosphere a residue from the means for heating the sample in an inert atmosphere can comprise an optical sensor operating in the ultraviolet range, the optical sensor comprising a source of UV radiation for illuminating a measurement area and a spectrometer for measuring the intensity as a function of the wavelength of the UV radiation that has passed through the measurement area.

The invention further concerns a method for quantifying a total nitrogen content in at least one of porous medium and biomass sample, characterized in that the method comprises at least the following steps:
heating the sample in an inert atmosphere, between a first temperature ranging between 50 °C and 300 °C and a second temperature ranging between 650 °C and 1000 °C, by following a first temperature sequence, continuously measuring at least an amount of NH₃, an amount of NO, an amount of NO₂, an amount of N₂O and an amount of N₂ released during the heating in an inert atmosphere, and deducing therefrom a NH₃ content, a N₂O content, a N₂ content, a first intermediate NO content and a first intermediate NO₂ content,
continuously oxidizing, by heating in an oxidizing atmosphere at a third temperature at least equal to 900 °C, at least part of the effluents from the heating of the sample in an inert atmosphere, continuously measuring at least an amount of NO and an amount of NO₂ released during the continuous oxidation of the part of the effluents from the heating in an inert atmosphere, and deducing therefrom a second intermediate NO content and a second intermediate NO₂ content,
heating in an oxidizing atmosphere a residue of the sample from the heating in an inert atmosphere between a fourth temperature ranging between 50 °C and 300 °C and a fifth temperature greater than or equal to 850 °C, by following a second temperature sequence, continuously measuring at least an amount of NO and an amount of NO₂ released during the heating of the residue in an oxidizing atmosphere, and deducing therefrom a third intermediate NO content and a third intermediate NO₂ content, and in that at least a total nitrogen content in the sample is determined from the sum of the NH₃, N₂O and N₂ contents, the first, second and third intermediate NO contents and the first, second and third intermediate NO₂ contents.

According to an implementation of the invention, the first temperature sequence can comprise a first isothermal stage, of a first predetermined duration, at the first temperature, and a second isothermal stage, of a second predetermined duration, at the second temperature, the first and second isothermal stages being connected by a first thermal gradient.

According to an implementation of the invention, the second temperature sequence can comprise a third isothermal stage, of a third predetermined duration, at the fourth temperature, and a fourth isothermal stage, of a fourth predetermined duration, at the fifth temperature, the third and fourth isothermal stages being connected by a second thermal gradient.

According to an implementation of the invention, the at least one of the first and second and third and fourth predetermined durations can range between 1 and 10 minutes, and the first and/or second thermal gradients can range between 15 °C/min and 40 °C/min.

According to an implementation of the invention, the method can be implemented by means of the device according to any of the variants described above.

### BRIEF DESCRIPTION OF THE DRAWINGS

Other features and advantages of the device and of the method according to the invention will be clear from reading the description hereafter of embodiments given by way of non-limitative example, with reference to the accompanying figures wherein:

FIG. 1 illustrates an example embodiment of the first temperature sequence implemented for the first step of the method according to the invention;

FIG. 2 illustrates an example embodiment of the second temperature sequence implemented for the third step of the method according to the invention;

FIG. 3 illustrates an example of implementation of the device according to the invention;

FIG. 4 illustrates a variant embodiment of the device according to the invention;

FIG. 5 shows the evolution of the sum of NO and NO₂ released as a function of temperature while carrying out the first step of the method according to the invention, in the case of a first sample type; and

FIG. 6 shows the evolution of the sum of NO and NO₂ released as a function of time while carrying out the first step of the method according to the invention, as well as the temperature sequence used for this step, in the case of a second sample type.

### DETAILED DESCRIPTION

The invention concerns a device and a method for quantifying a total nitrogen content in At least one of a porous medium and biomass sample.

What is understood by total nitrogen is both the organic nitrogen and the inorganic (or mineral) nitrogen content in the sample considered.

What is meant by porous medium sample is a sample of any consolidated or unconsolidated porous medium. It may be, by way of non-limitative example, a sample of a soil, of recent marine sediments, of a fertilizer or of a sedimentary rock. It may however also be (dried) sewage plant sludge, dredged river or harbor sediments. It may also be wet soils (marsh, mangroves).

Biomass is understood to be any mass of living material (animal, plant, earth, aquatic) remaining at equilibrium on a given surface of the earth. It may be, by way of non-limitative example, plants, biowaste or residual organic residues (including all the organic waste and by-products resulting from human and agricultural activities).

The porous medium or biomass sample, previously sifted (particle size less than or equal to 2 mm), dried and homogeneously crushed (particle size less than or equal to 0.25 mm) can have a mass of between 1 and 100 mg. Indeed, the method according to the invention, notably if it is implemented using the device according to the invention, does not require high-mass samples due to the sensitivity of the detectors and to the optimization of the circuits used. Besides, it is not necessary to pretreat the sample (in particular, no decarbonation, purification or extraction), notably with solvents.

In general terms, the method according to the invention is based on the measurement over time of all the nitrogen compounds contained in effluents released while heating the sample in an inert atmosphere, including the nitrogen compounds contained in an oxidation of the effluents released while heating the sample in an inert atmosphere, and on the measurement of all the nitrogen compounds contained in effluents released while heating in an oxidizing atmosphere a residue of the sample after heating in an inert atmosphere. In general, the nitrogen species produced by thermal cracking of all the nitrogen-containing chemical families contained in at least one of a porous medium and biomass sample are: NH₃, NO, NO₂, N₂O and N₂.

More precisely, the method according to the invention comprises at least the following steps:
1. Heating the sample in an inert atmosphere
2. Oxidizing part of the effluents resulting from heating in an inert atmosphere
3. Heating in an oxidizing atmosphere the residue resulting from heating in an inert atmosphere
4. Determining a total nitrogen content of the sample.

The device according to the invention comprises means for heating the sample in an inert atmosphere, means for oxidation of the effluents released while heating the sample in an inert atmosphere, means for heating a residue of the sample in an oxidizing atmosphere, and means for continuous measurement of the amounts of NH₃, NO, NO₂, N₂O and N₂ released while heating the sample in an inert atmosphere, means for continuous measurement of the amounts of NO and NO₂ released during oxidation of the effluents released while heating the sample in an inert atmosphere, and means for continuous measurement of the amounts of NO and NO₂ released while heating a residue of the sample in an oxidizing atmosphere. The method according to the invention can be advantageously implemented using the device according to the invention.

The steps of the method according to the invention are detailed hereafter.

### 1. Heating the Sample in an Inert Atmosphere

In this step, the sample is heated in an inert atmosphere, between a first temperature (referred to as T1 hereafter) ranging between 50 °C and 300 °C and a second temperature (referred to as T2 hereafter) ranging between 650 °C and 1000 °C, by following a first temperature sequence, an amount of NH₃, an amount of NO, an amount of NO₂, an amount of N₂O and an amount of N₂ released during the heating in an inert atmosphere are continuously measured, and a NH₃ content, a N₂O content, a N₂ content, a first intermediate NO content and a first intermediate NO₂ content are deduced therefrom.

In other words, in this step, the sample is pyrolyzed in a reducing medium, according to a temperature sequence whose lower and upper bounds correspond to temperatures T1 and T2 according to the invention. Indeed, the applicant was able to observe, by tests carried out on many types of porous media and biomass samples, that pyrolysis in an inert atmosphere, performed between these two temperatures T1 and T2, allows release, in form of effluents, all of the nitrogen compounds pyrolyzable in a reducing medium contained in a sample. Depending on the type of sample considered, the amounts of nitrogen and the thermal stability of the compounds that contain it can be very variable, from thermally unstable (volatile compounds) to thermally very stable. These temperature ranges allow better discrimination of the various thermal stability groups of the nitrogen compounds generated; the signals have higher resolution. Moreover, these tests show that the following nitrogen compounds can be released during this step: NH₃, NO, NO₂, N₂O and N₂. According to the invention, the following nitrogen compound amounts are continuously measured (i.e. during the inert-atmosphere heating time): an amount of NH₃, an amount of NO, an amount of NO₂, an amount of N₂O and an amount of N₂. A NH₃ content, a N₂O content, a N₂ content, a first intermediate NO content and a first intermediate NO₂ content are deduced from the measured NH₃, NO, NO₂, N₂O and N₂ contents. The reference here is to “intermediate contents” for NO and NO₂ nitrogen compounds because complementary contents are determined after steps 2 and 3 of the method according to the invention. In other words, the total NO and NO₂ content is not determined in this single step, unlike the NH₃, N₂O and N₂ nitrogen compound contents. It is clear that one or more of the measured nitrogen compound amounts can be zero, depending notably on the nature of the sample considered. Indeed, some porous media or biomass samples may release no NH₃ and/or NO and/or NO₂ and/or N₂O and/or N₂ during the first step of the method according to the invention.

According to an implementation of the invention, the temperature sequence used for carrying out this step can comprise a first isothermal stage at temperature T1 (i.e. temperature T1 is maintained for a first predetermined period of time) and a second isothermal stage at temperature T2 (i.e. temperature T2 is maintained for a second predetermined period of time), the two isothermal stages being connected by a thermal gradient (i.e. the first isothermal stage at temperature T1 is followed by a linear temperature increase up to the second isothermal stage at temperature T2).

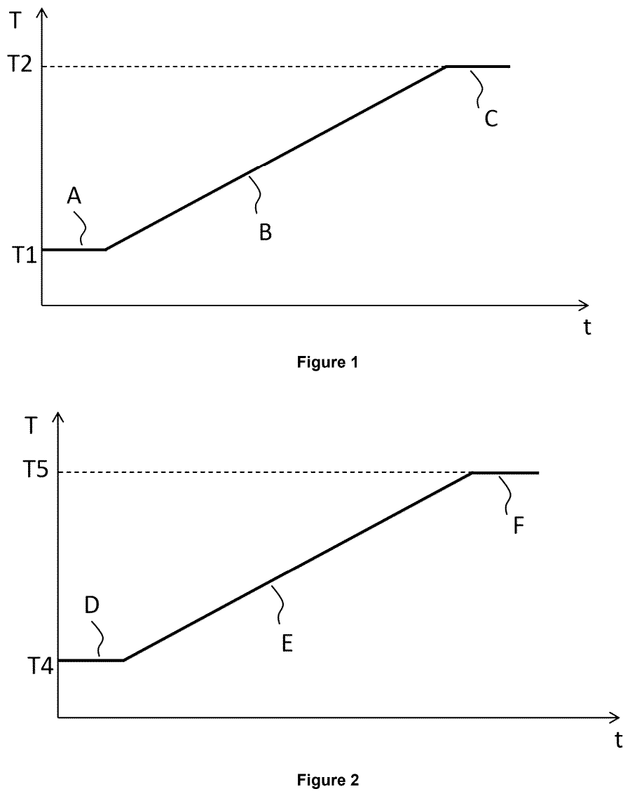

FIG. 1 schematically illustrates, by way of non-limitative example, an example embodiment of the first temperature sequence, carried out for the first step of the method according to the invention. More precisely, this example of a first temperature sequence comprises a first isothermal stage A at temperature T1, a second isothermal stage C at temperature T2, the two isothermal stages A and C being connected by a thermal gradient B.

According to an implementation of the invention, the first predetermined period of time of the stage at at least one of temperature T1 and the second predetermined period of time of the stage at temperature T2 can range between 1 and 10 min. Such durations allow release all the pyrolyzable nitrogen compounds present in a sample, while distinguishing between the lighter nitrogen compounds thermovaporizable (of low molecular weight) during temperature T1 and those obtained by cracking by pyrolysis, which are increasingly heavier (medium to high molecular weight) as the temperature increases according to the thermal gradient and up to the end of temperature T2. Advantageously, the thermal gradient can range between 15 °C/min and 40 °C/min. Such a gradient indeed allows to release all of the pyrolyzable nitrogen compounds present in the sample, within a time range of between 15 and 75 minutes according to the implementation, which is a short duration compared with the methods of the prior art, in particular compared with the chemical methods.

According to another implementation of the invention, the temperature sequence used for this step can comprise a first isothermal stage at temperature T1 (i.e. temperature T1 is maintained for a first predetermined period of time of between 1 and 10 minutes for example), a second isothermal stage at temperature T2 (i.e. temperature T2 is maintained for a second predetermined period of time of between 1 and 10 minutes for example), as well as a plurality of intermediate isothermal stages (for example 4 intermediate isothermal stages of between 1 and 10 minutes for example) between the first and the second stage, the stages being connected by thermal gradients (of between 15 °C/min and 40 °C/min for example). Such a sequence allows separation of even more precisely the thermovaporizable lighter nitrogen compounds from the nitrogen compounds thresholded between different temperature ranges in connection with different nitrogen-containing chemical classes from thermal cracking start to thermal cracking end of the last temperature stage. The same total nitrogen amount is obtained with this multi-stage sequence while having a higher resolution of the various signals providing better information on the different thermal classes of the nitrogen species present in the sample.

According to an implementation of the invention, the temperature sequence used for this step can comprise a succession of at least six isothermal stages of predetermined duration, including a first isothermal stage at temperature T1 of between 80 °C and 200 °C here, a first intermediate isothermal stage at a temperature of between 340 °C and 380 °C, a second intermediate isothermal stage at a temperature of between 400 °C and 440 °C, a third isothermal stage at a temperature of between 450 °C and 490 °C, a fourth intermediate isothermal stage at a temperature of between 500 °C and 540 °C, and a second isothermal stage at temperature T2, equal to 650 °C, the isothermal stages, intermediate or non intermediate, being connected by a thermal gradient. Advantageously, for this embodiment, it is also possible to continuously measure an amount of hydrocarbon compounds, an amount of CO and an amount of CO₂ released during this heating sequence.

According to an implementation of the invention, the inert gas used for this step can be helium He or argon Ar. It is clear that dinitrogen N₂ is advantageously avoided for implementing this step in order not to distort the measurements performed. In other words, in this step, the sample is heated in an inert atmosphere using an inert gas, to the exclusion of dinitrogen.

According to the invention, an amount of NH₃, an amount of NO, an amount of NO₂, an amount of N₂O and an amount of N₂ released during heating in an inert atmosphere are measured. For each nitrogen compound, it is then possible to generate a curve showing the evolution of the amount of this nitrogen compound over time, relative to the weight of the sample considered. In general, the applicant has observed that the curves representative of the evolution over time of the amount of each nitrogen compound released in steps 1, 2 or 3 have one or more peaks.

According to an implementation of the invention, for each nitrogen compound, from the (surface) area beneath the curve representative of the evolution over time of the amount of this nitrogen compound released during a given step (step 1, 2 or 3), a content can be determined for this nitrogen compound released during the step considered. This determination can notably be performed using the means for determining a total nitrogen content according to the invention, such as computer means. Any nitrogen compound selected from among the following list: NH₃, NO, NO₂, N₂O and N₂ is denoted by Nx hereafter.

According to an implementation of the invention, whatever the step considered (step 1, 2 or 3), the amount N_{Nx} in a nitrogen compound Nx can be determined according to a formula of the type:

$$\% wt . {N}_{Nx} = \frac{{M}_{N}}{{M}_{Nx}} \cdot {F}_{calib} \_Nx \cdot {Surface\_Nx}_{ech} \qquad ( 1 )$$

where % wt. N_{Nx} is the amount (in mass percent) of nitrogen in nitrogen compound Nx, M_{N} is the molar mass of nitrogen (M_{N}=14 g·mol⁻¹), M_{Nx} is the molar mass of the hydrogen compound Nx considered (for example M_{N0}=30 g·mol⁻¹), Surface_Nx_{ech} is the area beneath the curve representative of the evolution over time of the amount of nitrogen compound Nx released during the step considered (1, 2 or 3), and F_{calib}_Nx is a calibration factor for nitrogen compound Nx determined on a reference sample.

According to an implementation of the invention, calibration factor F_{calib}_Nx associated with nitrogen compound Nx can be determined from at least one reference sample whose nitrogen compound Nx content(s) is (are) known, the sample being subjected to the step considered (i.e. step 1, 2 or 3). The calibration coefficient is then determined from the area beneath the curve representative of the evolution over time of the amount of nitrogen compound(s) Nx released by this reference sample during the step considered, according to a formula of the type:

$${F}_{calib} \_Nx = \frac{m\_ref \_Nx}{{Surface\_Nx}_{ref}} . \qquad ( 2 )$$

where m_ref_Nx is the mass of the reference sample for nitrogen compound Nx, Surface_Nx_{ref} is the area beneath the curve representative of the evolution over time of the amount of nitrogen compound Nx released by the reference sample during the step considered. According to an implementation, the reference sample can be a porous medium or biomass sample whose nitrogen compound content(s) is (are) known, obtained for example by meansuse of an elemental analysis and of a modified Kjeldahl method. Alternatively, in the absence of a solid sample, the reference sample can be one or more nitrogen compounds Nx only, for example in form of a mixture or not of pure gases.

Thus, at the end of this step, there is a NH₃ content, a N₂O content, a N₂ content, a first intermediate NO content and a first intermediate NO₂ content.

2. Oxidizing Part of the Effluents Resulting from Heating in an Inert Atmosphere

This step continuously oxidizes at least part of the effluents (fluids) resulting from heating the sample considered in an inert atmosphere, by heating in an oxidizing atmosphere at a temperature at least equal to 900 °C (denoted by T3 hereafter), in continuously measuring an amount of NO and an amount of NO₂ released during this continuous oxidation of the part of the effluents from the heating in an inert atmosphere, and in deducing therefrom a second intermediate NO content and a second intermediate NO₂ content. According to an implementation of the invention, the oxidation can be catalytic oxidation.

In other words, in this step, at least part of the effluents resulting from pyrolysis in an inert atmosphere is subjected to oxidation. The purpose of this step is to quantify the nitrogen compounds contained in the heavier effluents generated during the pyrolysis phase in a reducing medium, by use of a high-temperature oxidation (T3 greater than or equal to 900 °C). The applicant has observed, by use of tests carried out on many different sample types, that only NO and NO₂ nitrogen compounds can be released in this step. Thus, according to the invention, an amount of NO and an amount of NO₂ released during this oxidation of the pyrolysis effluents are continuously measured. For each nitrogen compound released during this step, it is then possible to generate a curve showing the evolution of the amount of nitrogen compound considered, relative to the weight of the sample considered, as a function of time. Finally, a second intermediate NO content and a second intermediate NO₂ content are deduced from the NO and NO₂ amounts measured during this step. This determination can notably be performed using means for determining a total nitrogen content according to the invention, such as a computer. We refer here to “intermediate contents” because complementary contents are determined at the end of steps 1 and 3 of the method according to the invention. It is clear that the NO amount and the NO₂ amount measured in this step can be zero, depending on the nature of the sample being considered. Indeed, some porous medium or biomass samples may release no NO and NO₂ during the second step of the method according to the invention.

According to an implementation of the invention, for each of the NO and NO₂ nitrogen compounds released in step 2, from the area beneath the curve representative of the evolution over time of the nitrogen compound amount released in step 2, the intermediate amount of this nitrogen compound released in this step can be determined using the general formula of Equation (1) described in step 1.

According to an implementation of the invention, this step can be carried out using an oxidation oven heated to at least 900 °C and swept by a gas mixture comprising between 30% and 100% O₂ and up to 70% inert gas (He or Ar for example), with a flow rate of between 50 and 400 ml/min for example. According to an implementation of the invention, this step can be carried out using a catalytic oxidation oven.

According to an implementation of the invention, heating in an oxidizing atmosphere at least part of the effluents from heating of the sample considered in an inert atmosphere can be carried out with a heating sequence comprising a single isothermal stage at third temperature T3 of at least 900 °C (temperature T3), for a duration at least equal to the duration of step 1.

Thus, at the end of this step, a second intermediate NO content and a second intermediate NO₂ content are obtained.

3. Heating in an Oxidizing Atmosphere the Residue Resulting from Heating in an Inert Atmosphere

This step heats in an oxidizing atmosphere a (solid) sample residue from heating in an inert atmosphere between a fourth temperature (referred to as T4 hereafter) of between 50 °C and 300 °C and a fifth temperature (referred to as T5 hereafter) greater than or equal to 850 °C, by following a second temperature sequence, then an amount of NO and an amount of NO₂ released during the heating of the residue in an oxidizing atmosphere are continuously measured, and a third intermediate NO content and a third intermediate NO₂ content are deduced therefrom.

Thus, in this step, the (solid) residue from step 1 is subjected to oxidation according to a temperature sequence whose lower and upper bounds correspond to temperatures T4 and T5 according to the invention. The purpose of this step is to release, in the form of an oxidized gas, all the non-pyrolyzable (under the conditions of step 1) nitrogen compounds contained in the sample. The applicant has observed, by use of tests carried out on many different sample types, that only the following nitrogen compounds can be released while heating the pyrolysis residue in an oxidizing atmosphere: NO and NO₂. According to the invention, the released NO and NO₂ amounts are then continuously measured (measurement while heating the residue in an oxidizing atmosphere). Then, from the NO and NO₂ amounts measured in this step, a third intermediate NO content and a third intermediate NO₂ content are deduced therefrom. We refer here to “intermediate contents” because complementary contents are determined at the end of steps 1 and 2 of the method according to the invention, as described above. It is clear that at least one of the NO amount and the NO₂ amount measured in this step can be zero, depending on the nature of at least one of the sample considered and on the characteristics of the temperature sequence of step 1. Indeed, some porous medium or biomass samples may release no NO and/or no NO₂ during the third step of the method according to the invention. Moreover, at least one of the measured NO amount and NO₂ amount may also be zero when temperature T2 of the first step of the method according to the invention is very high (1000 °C for example). Indeed, in this case, all the pyrolyzable nitrogen compounds may have been already released during the first step of the method according to the invention.

According to an implementation of the invention, the temperature sequence used for this step can comprise a first isothermal stage at temperature T4 (i.e. temperature T4 is maintained for a fourth predetermined period of time) and a second isothermal stage at temperature T5 (i.e. temperature T5 is maintained for a fifth predetermined period of time), with the two isothermal stages being connected by at least one thermal gradient that is the isothermal stage at temperature T4 is followed by a linear temperature increase up to the isothermal stage at temperature T5. According to an implementation of the invention, the fourth predetermined period of time of the stage of at least one at temperature T4 and the fifth predetermined period of time of the stage at temperature T5 can range between 1 and 10 min. Such durations provide often incomplete combustion (NO and NO₂) of the organic nitrogen compounds still present in the pyrolysis residue depending on the combustion kinetics thereof. Advantageously, the thermal gradient can range between 15 °C/min and 40 °C/min. Such a gradient indeed allows release of all of the non-pyrolyzable (under the conditions of step 1) nitrogen compounds contained in the sample residue, and in a limited time (between 20 and 30 minutes depending on the operating conditions, which is short compared with the methods of the prior art). This fast temperature increase allows the combustion kinetics of very refractory effluents to be accelerated.

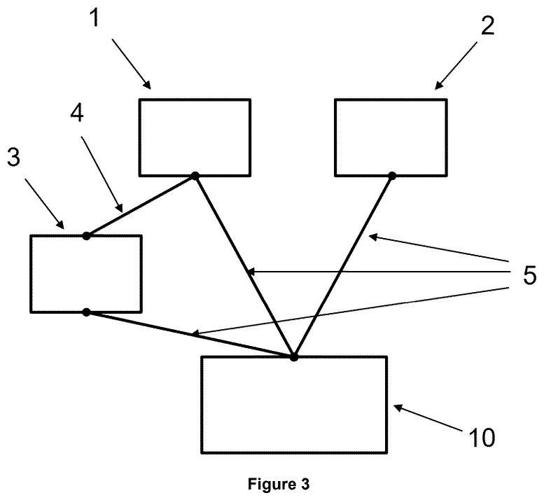

FIG. 2 schematically illustrates, by way of non-limitative example, an example embodiment of the second temperature sequence, carried out for the first step of the method according to the invention. More precisely, this second temperature sequence example comprises a first isothermal stage D at temperature T4, a second isothermal stage F at temperature T5, the two isothermal stages D, F being connected by a thermal gradient E.

According to the invention, an amount of NO and an amount of NO₂ released while heating the sample residue in an oxidizing atmosphere are measured. For each nitrogen compound released in this step, a curve showing the evolution of the amount of this nitrogen compound over time, relative to the weight of the sample considered, can then be generated.

According to an implementation of the invention, for each nitrogen compound, from the peak area (or from the area beneath the curve) of the curve representative of the amount of a nitrogen compound released in step 1, it is possible to determine the amount of a nitrogen compound Nx released in this step using the general formula of Equation (1) described in step 1. This determination can notably be performed using the means for determining a total nitrogen content according to the invention, such as a computer.

According to an implementation of the invention, temperature T4 can range between 150 °C and 300 °C, and temperature T5 can range between 850 °C and 1200 °C. Advantageously, a NO amount and a NO₂ amount released during this heating sequence can further be continuously measured.

Thus, at the end of this step, a third intermediate NO content and a third intermediate NO₂ content are obtained.

### 4. Determining a Total Nitrogen Content of the Sample

This step determines at least an amount of total nitrogen present in the sample from the NH₃, N₂O and N₂ contents determined in step 1, from the first, second and third NO contents determined in steps 1, 2 and 3 respectively, and from the first, second and third NO₂ contents determined in steps 1, 2 and 3 respectively.

In other words, in this step, the total nitrogen content is determined by the sum of all the contents determined in steps 1, 2 and 3. It is indeed the total nitrogen content because all the nitrogen compounds identified as released during steps 1, 2 and 3 are taken into account after several tests carried out on many porous medium and biomass sample types. As a reminder, steps 1 and 2 measure the pyrolyzable nitrogen content, whereas step 3 measures the non-pyrolyzable nitrogen under the conditions of step 1. This determination can notably be performed using means for determining a total nitrogen content according to the invention, such as a computer.

According to an advantageous implementation of the method according to the invention, and if an amount of hydrocarbon compounds, an amount of CO and an amount of CO₂ released during the first step carried out as described above, and an amount of CO and an amount of CO₂ released during the third step carried out as described above have further been continuously measured, it is further possible to characterize and to quantify the carbon present in the sample considered, by applying for example the method described in patent application WO-2022/200,091.

The invention further relates to a device for quantifying an amount of total nitrogen present in a porous medium and/or biomass sample, comprising:
means for heating the sample in an inert atmosphere,
means for heating in an oxidizing atmosphere at least part of the effluents from the means for heating the sample in an inert atmosphere,
means for heating in an oxidizing atmosphere a residue from the means for heating the sample in an inert atmosphere,
means suited for continuous measurement of:
   an amount of NH₃, an amount of NO, an amount of NO₂, an amount of N₂O and an amount of N₂ from the means for heating the sample in an inert atmosphere;
   an amount of NO and an amount of NO₂ from the means for oxidizing effluents from the means for heating the sample in an inert atmosphere; and
   an amount of NO and an amount of NO₂ from the means for heating in an oxidizing atmosphere a residue from the means for heating the sample in an inert atmosphere.

According to a first design of the device according to the invention, the means for heating a sample of the medium in an inert atmosphere can comprise a pyrolysis oven, the means for heating in an oxidizing atmosphere a residue from the means for heating the sample in an inert atmosphere can comprise a first oxidation oven, and the means for heating in an oxidizing atmosphere at least part of the effluents from the means for heating the sample in an inert atmosphere can comprise a second oxidation oven, which may be catalytic. Thus, with this design, the device comprises three distinct ovens. The pyrolysis oven can be an oven swept by an inert gas stream, such as helium He or argon Ar, at a flow rate of between 50 and 300 ml/min. It is clear that dinitrogen N₂ can be advantageously avoided so as not to distort the measurements performed.

The first oxidation oven can be swept by a gas mixture consisting of between 20% and 90% O₂, and up to 80% of an inert gas (for example He or Ar, and the inert gas does preferably not correspond to N₂), with a flow rate of between 50 and 200 ml/min for example. The second oxidation oven can be swept by pure oxygen to allow heating in a super-oxidizing atmosphere at a temperature at least equal to 900 °C, with a flow rate of between 50 and 400 ml/min. In this design, the device can comprise means for circulating the effluents released by the pyrolysis oven towards the measuring means suited for continuous measurement of at least an amount of NH₃, NO, NO₂, N₂O and N₂, in form of pipes through which an inert carrier gas such as helium He or argon Ar flows, and the inert gas does preferably not correspond to N₂. In this design, the device can further comprise means for circulating the effluents from the first oxidation oven towards the measuring means suited for continuous measurement of at least an amount of NO and of NO₂, in form of pipes through which a gas mixture comprising between 20% and 90% O₂, preferably 20% O₂, and between 10% and 80% of an inert gas, preferably 80% inert gas (for example He or Ar, and the inert gas does preferably not correspond to N₂) flows. In this design, the device can further comprise means for circulating the effluents from the second oxidation oven towards the measuring means suited for continuous measurement of at least an amount of NO and NO₂ in form of pipes through which a pure gas O₂ flows.

According to an implementation of the invention, at least one of the ovens of the device according to the invention can be made of stainless steel, alumina, porcelain. Such materials are suited to withstand the high temperatures used in the method according to the invention.

According to an implementation of the invention, the device can further comprise a boat intended to receive at least one of the porous medium and biomass sample. The boat can for example be made of stainless steel, alumina or porcelain, and it can be fed into the at least one of a pyrolysis oven and the oxidation oven by use of a piston for example.

In an alternative design of the device according to the invention, at least one of the means for heating a porous medium and biomass sample in an inert atmosphere and the means for heating in an oxidizing atmosphere a residue from the means for heating the sample in an inert atmosphere can comprise a single pyrolysis oven that can operate either in an inert atmosphere or in an oxidizing atmosphere, and the means for heating in an oxidizing atmosphere at least part of the effluents from the means for heating the sample in an inert atmosphere can comprise an oxidation oven (distinct from the pyrolysis oven that can operate either in an inert atmosphere or in an oxidizing atmosphere). In this configuration, the oxidation oven, which may be catalytic, distinct from the pyrolysis oven, allows continuous oxidation of the effluents from the means for heating in an inert atmosphere, that is, as they are being released while heating in an inert atmosphere.

According to a preferred implementation of the invention, the means for measuring suited for continuous measurement of an amount of NO (respectively an amount of NO₂) from the means for heating the sample in an inert atmosphere, the measuring means suited for continuous measurement of an amount of NO (respectively an amount of NO₂) from the means for oxidizing effluents from the means for heating the sample in an inert atmosphere, and the means for measuring suited for continuous measurement of an amount of NO (respectively an amount of NO₂) from the means for heating in an oxidizing atmosphere a residue from the means for heating the sample in an inert atmosphere can correspond to a single detector that can at least detect and measure the NO (respectively the NO₂). This allows not multiplying the number of detectors since the NO and NO₂ detectors can then be used for carrying out the three steps of the method according to the invention.

According to a preferred implementation of the invention that can be combined with the previous one, the measuring means suited for continuous measurement of at least:
an amount of NH₃, an amount of NO, an amount of NO₂, an amount of N₂O and an amount of N₂ from the means for heating the sample in an inert atmosphere;
an amount of NO and an amount of NO₂ from the means for oxidizing effluents from the means for heating the sample in an inert atmosphere; and
an amount of NO and an amount of NO₂ from the means for heating in an oxidizing atmosphere a residue from the means for heating the sample in an inert atmosphere corresponding to a single detector suited for continuous measurement of the following nitrogen compounds: NH₃, NO, NO₂, N₂O and N₂.

FIG. 3 schematically illustrates, by way of non-limitative example, an example of implementation of the device according to the invention comprising three distinct ovens and a single detector suited to measure an amount of each of the various nitrogen compounds released by a sample. More precisely, the device according to this example of an embodiment of the device according to the invention comprises a pyrolysis oven 1 for heating a sample in an inert atmosphere, a first oxidation oven 2 for heating in an oxidizing atmosphere a residue of the sample from heating in an inert atmosphere (the transfer from the pyrolysis oven to the oxidation oven are not shown), and a second oxidation oven 3 for heating in an oxidizing oven the effluents from heating of the sample in an inert atmosphere. In this design, a pipe 4 connects pyrolysis oven 1 to second oxidation oven 3 to allow transfer of part of the effluents from heating in an inert atmosphere to the high-temperature oxidation oven. Pyrolysis oven 1, first oxidation oven 2 and second oxidation oven 3 are connected by pipes 5 to a detector 10 suited for measuring an amount of each of the various nitrogen compounds that can be released by a sample.

According to an implementation, the measuring means suited for continuous measurement of at least an amount of NH₃ from the means for heating the sample in an inert atmosphere, and the means for measuring suited for continuous measurement of an amount of NO and an amount of NO₂ from the means for heating the sample in at least one of an inert atmosphere and from the means for heating in an oxidizing atmosphere a residue from the means for heating the sample in an inert atmosphere can be an optical sensor operating in the ultraviolet (UV) range. According to an implementation of the invention, the optical sensor can comprise a source of UV radiation for illuminating a measurement area (intended to receive effluents from any of the steps of the method according to the invention) and a spectrometer for measuring the intensity as a function of the wavelength of the UV radiation that has passed through the measurement area. The person skilled in the art has thorough knowledge of methods for determining the amount of different chemical species from measurements performed with such an optical sensor. One may for example refer to the method described in patent application WO-2019/020,326 A1, which concerns a method for measuring the concentration in different chemical species contained in exhaust gas. According to an implementation of the invention, from the measurement of the intensity as a function of the wavelength of the UV radiation that has passed through the measurement area and an intensity as a function of the reference wavelength, it is possible to determine an absorbance as a function of the wavelength of the effluents contained in the measurement area. The concentration in each nitrogen compound present in the effluents considered can then be determined from the absorbance of the calculated effluents, and also from predetermined absorbance characteristics and information relative to the temperature (known in our method since the temperature of the ovens is known at any time) and the pressure (known in our method and equal to the atmospheric pressure) of each chemical species to be quantified.

According to an implementation of the invention that can be combined with the previous one, the measuring means suited for continuous measurement of an amount of N₂ can correspond to a micro chromatograph (or micro GC). A micro chromatograph has an injector, a molecular sieve and a micro TCD (thermal conductibility detector) detector. In this implementation, a volume of gas can be removed, an internal sampling pump, from the stream flowing through the line of the device according to the invention in order to be sent to the micro GC.

According to an implementation of the invention that can be combined with the previous one, the measuring means suited for continuous measurement of an amount of NH₃ can correspond to an electrochemical detector. The principle of an electrochemical detector is based on a redox reaction.

According to an implementation of the invention that can be combined with the previous one, the measuring means suited for continuous measurement of an amount of NO can correspond to an infrared (IR) detector or a chemiluminescence (CLD) analyzer. The principle of CLD is based on the production of light as a result of a chemical reaction such as, for example, a redox reaction between a reduced species and an oxidizer, such as oxygen for example. Here, the chemiluminescence is produced by the photons (light) emitted as a result of the reaction between the nitrogen monoxide (NO) and the ozone (O₃) detected by a photomultiplier tube. In general, an IR detector comprises a spectroscopy sensor reacting to the infrared radiation. Advantageously, the infrared detector can use a narrow-band spectral region corresponding to that of the NO gas.

According to an implementation of the invention that can be combined with the previous one, the measuring means suited for continuous measurement of a NO₂ amount can correspond to an infrared detector. Advantageously, the infrared detector can use a narrow-band spectral region corresponding to that of the NO₂ gas.

According to an implementation of the invention that can be combined with the previous one, the measuring means suited for continuous measurement of a N₂O amount can correspond to an infrared detector. Advantageously, the infrared detector can use a narrow-band spectral region corresponding to that of the N₂O gas.

According to an embodiment wherein the device comprises at least two distinct nitrogen compound detectors, these detectors can be arranged in series, i.e. the gas stream flows through the detectors one after the other, each one being suited to detect a specific nitrogen compound.

Advantageously, the device according to the invention can further comprise a flame ionization type detector (FID) for continuous measurement of at least one of an amount of hydrocarbon compounds and an infrared (IR) type detector for continuous measurement of an amount of CO and CO₂.

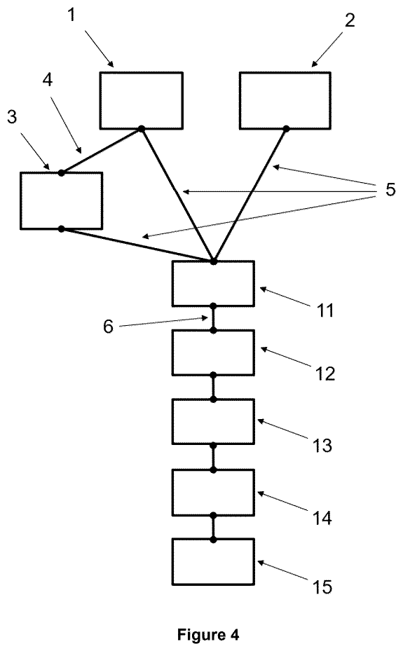

FIG. 4 schematically illustrates, by way of non-limitative example, a variant embodiment of the device according to the invention identical in every respect to the device of FIG. 3 (the common elements are therefore not described again), except that it comprises a detector specific to each of the various nitrogen compounds released by a sample, instead of a single detector. More precisely, the device according to this variant comprises five detectors 11, 12, 13, 14, 15 arranged in series, and each detector can specifically measure an amount of one of the following nitrogen compounds: NH₃, NO, NO₂, N₂O and N₂. The order in which detectors 11, 12, 13, 14, 15 are arranged has no incidence. Pyrolysis oven 1, first oxidation oven 2 and second oxidation oven 3 are connected by pipes 5 to first specific detector 11 of the series of detectors 11, 12, 13, 14, 15, and detectors 11, 12, 13, 14, 15 are connected two by two by pipes 6 having the same technical characteristics as pipes 5.

Thus, the device and the method according to the invention allow quantification of the total nitrogen present in at least one of a porous medium and a biomass sample in a single step, with no pretreatment, and using small amounts (from a few mg to a maximum of a hundred mg). The dynamic character (continuously measured signal) provides a second piece of information on the quality of the nitrogen constituents present in a sample according to the thermal stability thereof (continuous recording of the signal over time and temperature). These parameters allow better identification of the various mechanisms (N₂ fixation, nitrification, denitrification) involved during the carbon cycle-related respiration and fermentation processes. Besides, the present invention also provides, due to the detection of each nitrogen compound (by one or more detectors), information on the type of nitrogen compounds present in the sample (and not only concerning NO₂ or N₂ according to the analysis method used), while associating it with the thermal stability thereof.

### EXAMPLES

The features and advantages of the device and of the method according to the invention will be clear from reading the application example hereafter.

Samples containing variable nitrogen amounts (from 0.2 to 35% by mass) were analyzed with the method according to the invention, using the device according to the invention. The total nitrogen quantification results are comparable to the total nitrogen values measured by a reference analysis such as the elemental analysis according to the Dumas method.

More specifically, presented hereafter are the results of the embodiment of the method according to the invention using the device according to the invention, for a first example corresponding to a mineral fertilizer sample (KNO₃) and for a second example corresponding to a digested waste (digestate).

### First Example

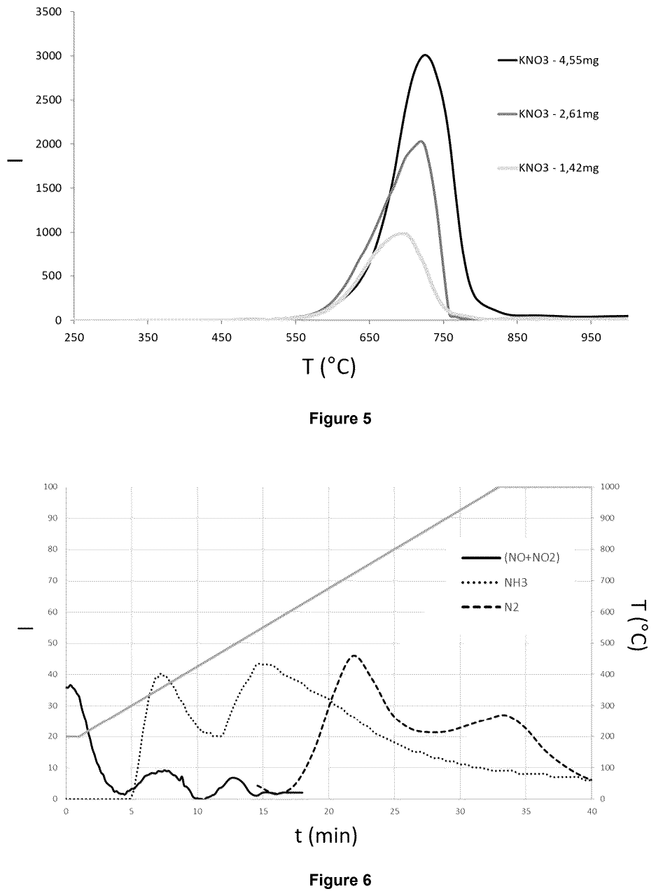

Applying the method according to the invention for the first example reveals that only the NO nitrogen compound is released in the case of a KNO₃ type nitrogen mineral. In other words, no N₂, NO₂, N₂O and NH₃ amount was measured in the method according to the invention. By way of example, FIG. 5 shows curves representing the evolution of the sum of NO and NO₂ released (through the intensity I of the measured signal) as a function of temperature T (the temperature being related to time via the temperature sequence) during the first step for KNO₃ samples having different masses: 4.55 mg (curve KNO₃-4.55 mg), 2.61 mg (curve KNO₃-2.61 mg) and 1.42 mg (curve KNO₃-1.42 mg). The first step of the method according to the invention was applied with a temperature sequence comprising a first isothermal stage at 250 °C maintained for 10 minutes, followed by a thermal gradient of 25 °C/min, up to a temperature of 100 °C maintained for 3 minutes. It is observed that the curves have similar geometries, but not the same intensity depending on the mass. Thus, the response of signal NO is linear with the mass of the sample to be analyzed.

Table 1 shows the total nitrogen amounts NT (in % by mass) obtained for the different sample masses, as well as the N₂, NO₂, N₂O and NH₃ amounts (zero quantities), and a total nitrogen value determined by an independent elemental analysis NAE. It is observed that the percentage by mass of total nitrogen, determined by applying the method according to the invention, is similar to the one determined by the elemental analysis, within 20%.

TABLE 1

| Sample | Nitrogen compound/Total nitrogen (% by mass) | | | | | | |
|---|---|---|---|---|---|---|---|
| mass (mg) | N—NO₂ | N—NO | N—NH₃ | N—N₂O | N—N₂ | NT | N_{AE} |

| | | | |
|---|---|---|---|
| 4.55 | 15.83 | 15.83 ± 2.8 | 13.85 |
| 2.61 | 17.1 | 17.1 ± 2.8 | 13.85 |
| 1.42 | 16.87 | 16.87 ± 2.8 | 13.85 |

### Second Example

Applying the method according to the invention to the second example shows that the following nitrogen compounds are released in the case of a digestate sample: NO, NO₂, NH₃ and N₂. FIG. 6 shows curves representing the evolution of the amount (through the intensity I of the measured signal) of cumulative NO and NO₂ (because the measured NO₂ signal was negligible; curve NO+NO₂), of NH₃ (curve NH₃) and of N₂ (curve N₂) released as a function of time t during the first step of the method according to the invention, as well as the temperature sequence used for this step (evolution of temperature T as a function of time t). It is observed that the various nitrogen compounds are not released at the same temperatures: notably NO and NO₂ are released at the lowest temperatures, whereas N₂ is released at the highest temperatures. Part of the NO/NO₂ signal also seems to be correlated with that of NH₃, indicating that these two gases result from cracking of the same chemical family. More than 90% of the NT content of the digestate comes from the N₂ signal. Table 2 shows the amounts (in % by mass) of total nitrogen NT, of N₂, NO₂, N₂O and NH₃ obtained with the method according to the invention, and the total nitrogen determined by an independent elemental analysis NAE. It is observed that the quantification obtained with the method according to the invention is in good agreement with that from the elemental analysis.

TABLE 2

| | Compound/Total nitrogen | | | | | |
|---|---|---|---|---|---|---|
| | N—(NO + NO₂) | N—NH₃ | N—N₂O | N—N₂ | NT | NT_{AE} |
| Measurement | 0.01 | 0.37 | | 7.9 | 8.28 ± 1.7 | 6.5 ± 0.34 |
| (% by mass) | | | | | | |

1-10. (canceled)

11. A device for quantifying total nitrogen content in at least one of a porous medium and a biomass sample, comprising:
means for heating the sample in an inert atmosphere;
means for heating in an oxidizing atmosphere a residue from the means for heating the sample in an inert atmosphere;
means for oxidizing effluents from the means for heating the sample in an inert atmosphere; and
means for continuously measuring an amount of NH₃, an amount of NO, an amount of NO₂, an amount of N₂O and an amount of N₂ from the means for heating the sample in an inert atmosphere, an amount of NO and an amount of NO₂ from the means for oxidizing effluents from the means for heating the sample in an inert atmosphere, and an amount of NO and an amount of NO₂ from the means for heating in an oxidizing atmosphere and a residue from the means for heating the sample in an inert atmosphere.

12. A device as claimed in claim 11, wherein the means for heating the sample in an inert atmosphere comprises an inert atmosphere pyrolysis oven, and the means for heating in an oxidizing atmosphere the residue from the means for heating the sample in an inert atmosphere comprises an oxidation oven.

13. A device as claimed in claim 11, wherein the means for heating the sample in an inert atmosphere and the means for heating in an oxidizing atmosphere the residue from the means for heating the sample in an inert atmosphere comprises a pyrolysis oven that operates in an inert atmosphere and in an oxidizing atmosphere.

14. A device as claimed in claim 11, wherein the means for oxidizing the effluents from the means for heating the sample in an inert atmosphere comprises an oxidation oven.

15. A device as claimed in claim 12, wherein the means for oxidizing the effluents from the means for heating the sample in an inert atmosphere comprises an oxidation oven.

16. A device as claimed in claim 13, wherein the means for oxidizing the effluents from the means for heating the sample in an inert atmosphere comprises an oxidation oven.

17. A device as claimed claim 11, wherein the means for continuous measurement of the amount of NH₃ from the means for heating the sample in an inert atmosphere, and the means for continuous measurement of at least one of the amount of NO and the amount of NO₂ from the means for heating the sample in at least one of an inert atmosphere and from the means for oxidizing effluents from the means for at least one of heating the sample in an inert atmosphere and from the means for heating in an oxidizing atmosphere a residue from the means for heating the sample in an inert atmosphere comprises an optical sensor operating in the ultraviolet range, comprising a source of UV radiation for illuminating a measurement area and a spectrometer for measuring the intensity as a function of the wavelength of the UV radiation that has passed through the measurement area.

18. A device as claimed claim 12, wherein the means for continuous measurement of the amount of NH₃ from the means for heating the sample in an inert atmosphere, and the means for continuous measurement of at least one of the amount of NO and the amount of NO₂ from the means for heating the sample in at least one of an inert atmosphere and from the means for oxidizing effluents from the means for at least one of heating the sample in an inert atmosphere and from the means for heating in an oxidizing atmosphere a residue from the means for heating the sample in an inert atmosphere comprises an optical sensor operating in the ultraviolet range, comprising a source of UV radiation for illuminating a measurement area and a spectrometer for measuring the intensity as a function of the wavelength of the UV radiation that has passed through the measurement area.

19. A device as claimed claim 13, wherein the means for continuous measurement of the amount of NH₃ from the means for heating the sample in an inert atmosphere, and the means for continuous measurement of at least one of the amount of NO and the amount of NO₂ from the means for heating the sample in at least one of an inert atmosphere and from the means for oxidizing effluents from the means for at least one of heating the sample in an inert atmosphere and from the means for heating in an oxidizing atmosphere a residue from the means for heating the sample in an inert atmosphere comprises an optical sensor operating in the ultraviolet range, comprising a source of UV radiation for illuminating a measurement area and a spectrometer for measuring the intensity as a function of the wavelength of the UV radiation that has passed through the measurement area.

20. A device as claimed claim 14, wherein the means for continuous measurement of the amount of NH₃ from the means for heating the sample in an inert atmosphere, and the means for continuous measurement of at least one of the amount of NO and the amount of NO₂ from the means for heating the sample in at least one of an inert atmosphere and from the means for oxidizing effluents from the means for at least one of heating the sample in an inert atmosphere and from the means for heating in an oxidizing atmosphere a residue from the means for heating the sample in an inert atmosphere comprises an optical sensor operating in the ultraviolet range, comprising a source of UV radiation for illuminating a measurement area and a spectrometer for measuring the intensity as a function of the wavelength of the UV radiation that has passed through the measurement area.

21. A device as claimed claim 15, wherein the means for continuous measurement of the amount of NH₃ from the means for heating the sample in an inert atmosphere, and the means for continuous measurement of at least one of the amount of NO and the amount of NO₂ from the means for heating the sample in at least one of an inert atmosphere and from the means for oxidizing effluents from the means for at least one of heating the sample in an inert atmosphere and from the means for heating in an oxidizing atmosphere a residue from the means for heating the sample in an inert atmosphere comprises an optical sensor operating in the ultraviolet range, comprising a source of UV radiation for illuminating a measurement area and a spectrometer for measuring the intensity as a function of the wavelength of the UV radiation that has passed through the measurement area.

22. A device as claimed claim 16, wherein the means for continuous measurement of the amount of NH₃ from the means for heating the sample in an inert atmosphere, and the means for continuous measurement of at least one of the amount of NO and the amount of NO₂ from the means for heating the sample in at least one of an inert atmosphere and from the means for oxidizing effluents from the means for at least one of heating the sample in an inert atmosphere and from the means for heating in an oxidizing atmosphere a residue from the means for heating the sample in an inert atmosphere comprises an optical sensor operating in the ultraviolet range, comprising a source of UV radiation for illuminating a measurement area and a spectrometer for measuring the intensity as a function of the wavelength of the UV radiation that has passed through the measurement area.

23. A method for quantifying a total nitrogen content in at least one of a porous medium and a biomass sample, comprising of:
heating the sample in an inert atmosphere, between a first temperature ranging between 50 °C and 300 °C and a second temperature ranging between 650 °C and 1000 °C, by following a first temperature sequence, continuously measuring at least an amount of NH₃, an amount of NO, an amount of NO₂, an amount of N₂O and an amount of N₂ released during the heating in an inert atmosphere, and deducing therefrom NH₃ content, N₂O content, N₂ content, a first intermediate NO content and a first intermediate NO₂ content;
continuously oxidizing, by heating in an oxidizing atmosphere at a third temperature at least equal to 900 °C, at least part of the effluents from the heating of the sample in an inert atmosphere, continuously measuring at least an amount of NO and an amount of NO₂ released during the continuous oxidation of the part of the effluents from the heating in an inert atmosphere, and deducing therefrom a second intermediate NO content and a second intermediate NO₂ content;
heating in an oxidizing atmosphere a residue of the sample from the heating in an inert atmosphere between a fourth temperature ranging between 50 °C and 300 °C and a fifth temperature greater than or equal to 850 °C, by following a second temperature sequence, continuously measuring at least an amount of NO and an amount of NO₂ released during the heating of the residue in an oxidizing atmosphere, and deducing therefrom a third intermediate NO content and a third intermediate NO₂ content; and
and determining in at least a total nitrogen content in the sample from a sum of the NH₃, N₂O and N₂, the first, second and third intermediate NO contents and the first, second and third intermediate NO₂ contents.

24. A method as claimed in claim 17, wherein the first temperature sequence comprises a first isothermal stage, of a first predetermined duration, at the first temperature, and a second isothermal stage, of a second predetermined duration, at the second temperature, and the first and second isothermal stages being connected by a first thermal gradient.

25. A method as claimed in claim 23, wherein the first temperature sequence comprises a first isothermal stage, of a first predetermined duration, at the first temperature, and a second isothermal stage, of a second predetermined duration, at the second temperature, and the first and second isothermal stages being connected by a first thermal gradient.

26. A method as claimed in claim 17, wherein the second temperature sequence comprises a third isothermal stage, of a third predetermined duration, at the fourth temperature, and a fourth isothermal stage, of a fourth predetermined duration, at the fifth temperature, and the third and fourth isothermal stages being connected by a second thermal gradient.

27. A method as claimed in claim 26, wherein at least one of the first, second, third and fourth predetermined durations range between 1 and 10 minutes, and at least one of the first and second thermal gradients range between 15 °C/min and 40 °C/min.

28. A method in accordance with claim 23, wherein the device comprises means for heating the sample in an inert atmosphere; means for heating in an oxidizing atmosphere a residue from the means for heating the sample in an inert atmosphere; means for oxidizing effluents from the means for heating the sample in an inert atmosphere; and means for continuously measuring an amount of NH₃, an amount of NO, an amount of NO₂, an amount of N₂O and an amount of N₂ from the means for heating the sample in an inert atmosphere, an amount of NO and an amount of NO₂ from the means for oxidizing effluents from the means for heating the sample in an inert atmosphere, and an amount of NO and an amount of NO₂ from the means for heating in an oxidizing atmosphere and a residue from the means for heating the sample in an inert atmosphere.
